SEQUENCE LISTING (11 sequences)

SEQ ID NO: 1 - DNA, 340 bp, Brassica napus
gtatcgaagatagtttgattttttggctctagataaaacattcatgcttaaaatatcgggaggttcttaacacaatagaaagttaaaaagagaatataggaaaattctcaattaagcacttttaagaaacaattacaatactgacacatgtcacctctttattggttctgtttttttaaagcaaagtaaaaagtaaatacattagtataatattaattttttttttcttttagaatctctcacatgttttcagccatgggtatgctcttataataaaaaaaaaaacataatcccatacacagccacatttgttgtttctccaaccaacctctcattataa

SEQ ID NO: 2 - DNA, 710 bp, Brassica napus
taggaagagagagagtaatattcagtagttacactgagagaattttagttcaccaattcaaatgtttaaaatgcataaattaaaactgagttgatttttaaaaaaaaatagtgtgtaaacaacattaaattatatgcgctggagttaaaaagaattagggtgtgaatacaataacttttaactaattatgagactgctccatttttattctaattttcaagttattgtgaaacttcccatgtcaattactgtttgaatacatttattataaatttttcttttttaatatgtaaagggtatcgaaaataagtttggtttgatatttttggttctagaaaaaacattcatgcttaaaaattaatataaaaataaattaaagtatggggtgtaaaagcatgtttatcagtaggttcttacacaaatttctaaaattaaaataatagaaggctaagaaagagaaaataggaaaattctcaattaaacactttttaagaacggttccaatactgacacacatatgtgtcacttacctctttattggttctgttttataaacaaaaaaaacagtaaatacattagtataatattaatatatttttttcttttagaatctctcgcatgttttcggccatgggcatggtattataataaaaaacataatcccatacacagccacatttcttgtttctccaaccaacctctcattataa

SEQ ID NO: 3 - DNA, 1546 bp, Brassica napus
tccagcggactacatgcaaagtggtagttgtcacaatgacgttgttttagctctcattacctttttttttgagctctcattgcttacttcacgtcttctaacaatatttgtttgctaccgacgttctactaaatcacaaaaataaacttaactgaacctattttgaccatatccacttgaaaaaactgtgaacaaaaaaagaagataaccaaagtaagatatggatgtacatgattggcccttatcccaatacatatggtatcagaaaagtttgtggcagttaaagttcatcagactgctgtactaacatcataatttcagacgcagtcacgtttctcgtctctccaacctccattgcaccgtccatcctaaaagagataatactaatttttttataaaaaatatgataatatattaatttagaattactctattttaaaataaaaaaatagagaaccattggaaatggtataagacggaaccactgatcactcatataaagctaccgaccatcaagaatgatatgcgaaagagaacaaccacgtaagtgaagcaggagaagtttatcaaaattttgaaggagaagtatcacagctaagagatgctggttcttaatctattggagaggaagatgaagaagagttttgtgttgaagagagaccatggtataccatactctgatcaacatgatgaaaaccaacaaaaaactcattatcaagtcgactaaaaaattatagaggagaacaagaatgccaacatatatttgtttgaagaaaagtcttcaatgaggttggaagagatgatgataagttcagatacatccattttgcagaccatcatcaagaaccataaagatacttatacggggagagaagcataaaacaaccagtttagatgtttttagatttttatgaattttatgattttctaaaactttatatctatggaaatttattattttatgaaatattcaattttttggaaaaagaacaactgtttttttgcaagagctgttgttaattgagaacattcataaaattgatgtactaagttgacaaaacagttaatggaattattatattaaataacagaaaggttaagtattaaatggcttataatttttttactttcttgtcaaagttcttataaaaatttagttggaatactgttataaaaaaaattaaatacatgttgatataaatatttggtttatcgattacattttagatatttactaattttaaaactaaatatatataaaatattaagagtaaaagacgtatttcaatatattcatgaatacattcaattttcagtttgattcgtgtccaatttttagatattgaaagcagaaactatttagatatttttgattattcagttaagtttggactgtttggtttgatttgtcggtcctaaataaaacatccttacctaaaaattaatataaagataaataaaaagtagaggactgtagcaataaagaatacataatccccctccatacacagagccactttcttgttccgccaacctctcattataa

SEQ ID NO: 4 - DNA, 477 bp, Brassica napus
gtatcgaagatagtttgattttttggctctagataaaacattcatgcttaaaatatcgggaggttcttaacacaatagaaagttaaaaagagaatataggaaaattctcaattaagcacttttaagaaacaattacaatactgacacatgtcacctctttattggttctgtttttttaaagcaaagtaaaaagtaaatacattagtataatattaattttttttttcttttagaatctctcacatgttttcagccatgggtatgctcttataataaaaaaaaaaacataatcccatacacagccacatttgttgtttctccaaccaacctctcattataaatgaaagcgactctcgcaccaccctcctctctcataagcctcccaaggcacaaagtatcttctctccgttcaccgtcgcttctccttcagtcccaacggccatcctcagccttaatgacaacgacggcatcacgtgg

SEQ ID NO: 5 - DNA, 137 bp, Brassica napus
atgaaagcgactctcgcaccaccctcctctctcataagcctcccaaggcacaaagtatcttctctccgttcaccgtcgcttctccttcagtcccaacggccatcctcagccttaatgacaacgacggcatcacgtgg

SEQ ID NO: 6 - DNA, 847 bp, Brassica napus
taggaagagagagagtaatattcagtagttacactgagagaattttagttcaccaattcaaatgtttaaaatgcataaattaaaactgagttgatttttaaaaaaaaatagtgtgtaaacaacattaaattatatgcgctggagttaaaaagaattagggtgtgaatacaataacttttaactaattatgagactgctccatttttattctaattttcaagttattgtgaaacttcccatgtcaattactgtttgaatacatttattataaatttttcttttttaatatgtaaagggtatcgaaaataagtttggtttgatatttttggttctagaaaaaacattcatgcttaaaaattaatataaaaataaattaaagtatggggtgtaaaagcatgtttatcagtaggttcttacacaaatttctaaaattaaaataatagaaggctaagaaagagaaaataggaaaattctcaattaaacactttttaagaacggttccaatactgacacacatatgtgtcacttacctctttattggttctgttttataaacaaaaaaaacagtaaatacattagtataatattaatatatttttttcttttagaatctctcgcatgttttcggccatgggcatggtattataataaaaaacataatcccatacacagccacatttcttgtttctccaaccaacctctcattataaatgaaagcgacactcgcaccaccctcctctctcataagcctccccaggcacaaagtatcttccctccgttcaccgtcgcttctccttcagtcccaacggcgatcctcagccttaatgacgacgacggcatcacgtgg

SEQ ID NO: 7 - DNA, 137 bp, Brassica napus
atgaaagcgacactcgcaccaccctcctctctcataagcctccccaggcacaaagtatcttccctccgttcaccgtcgcttctccttcagtcccaacggcgatcctcagccttaatgacgacgacggcatcacgtgg

SEQ ID NO: 8 - DNA, 1680 bp, Brassica napus
tccagcggactacatgcaaagtggtagttgtcacaatgacgttgttttagctctcattacctttttttttgagctctcattgcttacttcacgtcttctaacaatatttgtttgctaccgacgttctactaaatcacaaaaataaacttaactgaacctattttgaccatatccacttgaaaaaactgtgaacaaaaaaagaagataaccaaagtaagatatggatgtacatgattggcccttatcccaatacatatggtatcagaaaagtttgtggcagttaaagttcatcagactgctgtactaacatcataatttcagacgcagtcacgtttctcgtctctccaacctccattgcaccgtccatcctaaaagagataatactaatttttttataaaaaatatgataatatattaatttagaattactctattttaaaataaaaaaatagagaaccattggaaatggtataagacggaaccactgatcactcatataaagctaccgaccatcaagaatgatatgcgaaagagaacaaccacgtaagtgaagcaggagaagtttatcaaaattttgaaggagaagtatcacagctaagagatgctggttcttaatctattggagaggaagatgaagaagagttttgtgttgaagagagaccatggtataccatactctgatcaacatgatgaaaaccaacaaaaaactcattatcaagtcgactaaaaaattatagaggagaacaagaatgccaacatatatttgtttgaagaaaagtcttcaatgaggttggaagagatgatgataagttcagatacatccattttgcagaccatcatcaagaaccataaagatacttatacggggagagaagcataaaacaaccagtttagatgtttttagatttttatgaattttatgattttctaaaactttatatctatggaaatttattattttatgaaatattcaattttttggaaaaagaacaactgtttttttgcaagagctgttgttaattgagaacattcataaaattgatgtactaagttgacaaaacagttaatggaattattatattaaataacagaaaggttaagtattaaatggcttataatttttttactttcttgtcaaagttcttataaaaatttagttggaatactgttataaaaaaaattaaatacatgttgatataaatatttggtttatcgattacattttagatatttactaattttaaaactaaatatatataaaatattaagagtaaaagacgtatttcaatatattcatgaatacattcaattttcagtttgattcgtgtccaatttttagatattgaaagcagaaactatttagatatttttgattattcagttaagtttggactgtttggtttgatttgtcggtcctaaataaaacatccttacctaaaaattaatataaagataaataaaaagtagaggactgtagcaataaagaatacataatccccctccatacacagagccactttcttgttccgccaacctctcattataaatgaaagcgactctcgcaccctcctctctcataagcctccccaggcacaaagtatcttctctccgttcaccgtcgcttctccttcagtcccaacggccatcctcagccttaatgacgacgacggcatcacgtgg

SEQ ID NO: 9 - DNA, 134 bp, Brassica napus
atgaaagcgactctcgcaccctcctctctcataagcctccccaggcacaaagtatcttctctccgttcaccgtcgcttctccttcagtcccaacggccatcctcagccttaatgacgacgacggcatcacgtgg

SEQ ID NO: 10 - DNA, 27 bp, Artificial Sequence
  Gene specific primer
gtgatgcatatgatctccccaaatctc

SEQ ID NO: 11 - DNA, 27 bp, Artificial Sequence
  Gene specific primer
ccacgtgatgccgtcgtcgtcattaag

(19) United States
(12) Patent Application Publication
(10) Pub. No.: US 2003/0166205 A1
(43) Pub. Date: Sep. 4, 2003

(54) Plant regulatory sequences

(76) Inventors: Alison Van Eenennaam, Davis, CA (US); Eric Aasen, Woodland, CA (US); Charlene Levering, Davis, CA (US)

Correspondence Address: ARNOLD & PORTER, IP DOCKETING DEPARTMENT; RM 1126(b), 555 12TH STREET, N.W., WASHINGTON, DC, 20004-1206, US

(21) Appl. No.: 10/067,279

(22) Filed: Feb. 7, 2002

Related U.S. Application Data
(60) Provisional application No. 60/267,330, filed on Feb. 8, 2001.

(51) Int. Cl. C12N 9/10; C12Q 1/68; C07H 21/04; C12P 21/02; C12N 5/04

(52) U.S. Cl. 435/193; 435/6; 435/69.1; 435/419; 435/320.1; 536/23.2

(57) ABSTRACT

The present invention relates to the isolation of nucleic acid sequences upstream of the gamma-tocopherol methyltransferase (GMT) coding sequence in the genome of Brassica napus and the use of such sequences in methods to control gene expression of polypeptides, preferably GMT in plants. The present invention further pertains to methods of regulating expression of polypeptides using transcription factors, preferably zinc finger transcription factors and the isolated nucleic acid sequences upstream of the gene encoding GMT which contain binding sites for the transcription factors.

This application claims the benefit under 35 U.S.C. §119(e) of U.S. Provisional Patent Application Serial No. 60/267,330, filed Feb. 8, 2001, which application is herein incorporated by reference in its entirety.

A paper copy of the sequence listing and a computer readable form of the sequence listing on diskette, containing the filed named “16515.131seqlist.txt”, which is 9,003 bytes in size (measured in MS-DOS), and which was created on Feb. 4, 2002 are herein incorporated by reference.

### FIELD OF THE INVENTION

The present invention relates to the isolation of nucleic acid sequences upstream of the gamma-tocopherol methyltransferase (GMT) coding sequence in the genome of Brassica napus and the use of such sequences in methods to control gene expression of polypeptides, as preferably GMT in plants. The present invention further pertains to methods of regulating expression of polypeptides using transcription factors, preferably zinc finger transcription factors and the isolated nucleic acid sequences upstream of the gene encoding GMT which contain binding sites for the transcription factors.

### BACKGROUND OF THE INVENTION

One of the goals of plant genetic engineering is to produce plants with agronomically important characteristics or traits. Recent advances in genetic engineering have provided the requisite tools to transform plants to contain and express foreign genes (Kahl et al. (1995) World Journal of Microbiology and Biotechnology 11:449-460). The technological advances in plant transformation and regeneration have enabled researchers to take pieces of DNA, such as a gene or genes from a heterologous DNA, or native DNA modified to have different or improved qualities, and incorporate the exogenous DNA into the plant's genome. The gene or gene(s) can then be expressed in the plant cell to exhibit the added characteristic(s) or trait(s). In one approach, expression of a novel gene that is not normally expressed in a particular plant or plant tissue may confer a desired phenotypic effect. In another approach, transcription of a gene or part of a gene in an antisense orientation may produce a desirable effect by preventing or inhibiting expression of an endogenous gene.

The isolation of plant regulatory sequences is useful for modifying plants through genetic engineering to have desired phenotypic characteristics. In order to produce such a transgenic plant, a vector that includes a heterologous nucleotide sequence that confers the desired phenotype when expressed in the plant is introduced into the plant cell. The vector also includes a regulatory sequence that is operably linked to the heterologous nucleotide sequence, often a regulatory sequence not normally associated with the heterologous sequence. The vector is then introduced into a plant cell to produce a transformed plant cell, and the transformed plant cell is regenerated into a transgenic plant. The regulatory sequence controls expression of the introduced nucleotide sequence to which the regulatory sequence is operably linked and thus affects the desired characteristic conferred by the nucleotide sequence.

A variety of different types or classes of regulatory sequences can be used for plant genetic engineering. Regulatory sequences can be classified on the basis of range or tissue specificity. For example, regulatory sequences referred to as constitutive regulatory sequences are capable of transcribing operatively linked nucleotide sequences efficiently and expressing said nucleotide sequences in multiple tissues. Tissue-enhanced or tissue-specific regulatory sequences can be found upstream and operatively linked to nucleotide sequences normally transcribed in higher levels in certain plant tissues or specifically in certain plant tissues. Other classes of regulatory sequences can include, but are not limited to, inducible regulatory sequences that can be triggered by external stimuli such as chemical agents or environmental stimuli; temporally regulated regulatory sequences that are functional only or predominantly during certain periods of plant development or at certain times of day, as in the case of genes associated with circadian rhythm; and developmentally regulated regulatory sequences that are functional only at a certain period of plant development. Thus, regulatory sequences can be obtained by isolating the upstream 5′ regions of DNA sequences that are transcribed and expressed in a constitutive, tissue-enhanced, developmental or inducible manner.

Transcriptional activation of gene expression is primarily mediated through transcription factors that interact with enhancer and promoter elements of a regulatory site. Binding of transcription factors to such DNA elements constitutes a crucial step in transcriptional initiation. Structural and functional analyses of transcription factors reveal that many of these proteins have a modular protein structure, i.e., they are often modular, made up of a specific DNA-binding domain and a separate and independently acting activation domain. Researchers have found that heterogeneous domains can be combined, the resultant composite activators being functional in mammalian cells. An example of such an activator is the protein produced by fusion of the Gal4 DNA-binding domain with the activation domain of VP16. Each transcription factor binds to its specific binding sequence in a regulatory sequence, usually a promoter sequence and activates expression of the linked coding region through interactions with coactivators and/or proteins that are a part of the transcription complex.

Vitamin E is the term used to refer to a group of tocopherols and tocotrienols, of which alpha-tocopherol has the highest biological activity. Tocopherols have four members which are designated alpha, beta, gamma and delta tocopherol. Alpha tocopherol is largely considered the most important member of the class of tocopherols because it constitutes about 90% of the tocopherols found in animal tissues and is most readily absorbed and retained by the body. Furthermore, the in vivo antioxidant activity of alpha-tocopherol is higher than the antioxidant activities of beta, gamma and delta tocopherol.

Only plants and certain other photosynthetic organisms including cyanobacteria, synthesize tocopherols. The gamma-tocopherol methyltransferase (GMT) enzyme catalyzes the methylation of gamma-tocopherol to form alpha-tocopherol, the final step of alpha tocopherol biosynthesis. Overexpression of a gamma-tocopherol methyltransferase gene in a plant was reported to enhance the conversion of gamma-tocopherol to alpha-tocopherol (Shintani and DellaPenna, 1998). Certain gene sequences encoding GMT from photosynthetic organisms are set forth in PCT applications PCT/US98/15137 and PCT/US99/28588.

Accordingly, the identification and isolation of regulatory sequences capable of regulating expression of GMT in plant tissues is desirable in order to produce transgenic plants containing increased levels of alpha-tocopherol. Furthermore, the isolated regulatory sequences may be used for selectively modulating expression of any operatively linked gene and provide additional regulatory element diversity in a plant expression vector. There is also a need for identification of transcription factors under the control of a seed-specific promoter for use in conjunction with such isolated regulatory sequences in order to produce a GMT protein in the seed. The ability to increase production of GMT in a seed or plant will catalyze the conversion of gamma-tocopherol to alpha-tocopherol thus increasing the levels of alpha-tocopherol.

### SUMMARY OF THE INVENTION

Thus, one aspect of the present invention to provide isolated plant regulatory sequences that comprise nucleic acid regions located upstream of the gene encoding GMT.

One aspect of the invention is directed to nucleic acid sequences comprising any one of SEQ ID NOS: 1-3, fragments of SEQ ID NOS: 1-3, nucleic acid sequences having at least 80% homology to any one of SEQ ID NOS: 1-3, the complements of SEQ ID NOS: 1-3 and fragments of the complements of SEQ ID NOS: 1-3. Another related aspect of the present invention is the provision of such regulatory sequences that comprise at least one binding site for a transcription factor, preferably a zinc finger transcription factor.

The present invention includes an isolated nucleic acid molecule comprising a nucleic acid sequence selected from the group consisting of SEQ ID NO: 1, SEQ ID NO: 2, SEQ ID NO: 3 and complements thereof. The present invention also includes an isolated nucleic acid molecule comprising a nucleic acid sequence that is at least 30 consecutive nucleotides of a nucleic acid sequence selected from the group consisting of SEQ ID NO: 1, SEQ ID NO: 2, SEQ ID NO: 3 and complements thereof.

Another aspect provides methods of regulating expression of a polypeptide in a cell comprising introducing into a cell a vector comprising a nucleic acid molecule encoding a transcription factor preferably, a zinc finger transcription factor which binds to any one of SEQ ID NOS: 1-3 whereby the expression of the transcription factor regulates expression of the polypeptide in the cell.

It is a further aspect of the present invention to provide vectors, host cells and transgenic plants containing the nucleic acid sequences encoding for a transcription factor which bind to any one of SEQ ID NOS: 1-3, or any fragments, complements or regions thereof. It is another aspect of the present invention to provide vectors, host cells and transgenic plants containing the nucleic acid sequences as shown in SEQ ID NOS: 1-3, or any fragments, complements or regions thereof.

The present invention includes a vector comprising a nucleic acid molecule comprising a nucleic acid sequence selected from the group consisting of SEQ ID NO: 1, SEQ ID NO: 2, SEQ ID NO: 3 and complements thereof operably linked to polypeptide encoding nucleic acid sequence. The present invention also includes a vector comprising a nucleic acid molecule comprising a nucleic acid sequence selected from the group consisting of SEQ ID NO: 1, SEQ ID NO: 2, SEQ ID NO: 3 and complements thereof operably linked to a heterologous nucleic acid sequence in manner where the complement of said heterologous nucleic acid sequence is expressed.

The present invention further includes a host cell having a heterologous nucleic acid molecule that comprises a nucleic acid sequence selected from the group consisting of SEQ ID NO: 1, SEQ ID NO: 2, SEQ ID NO: 3 and complements thereof. The present invention also includes a host cell having a heterologous nucleic acid molecule that comprises a nucleic acid sequence that is at least 30 consecutive nucleotides of a nucleic acid sequence selected from the group consisting of SEQ ID NO: 1, SEQ ID NO: 2, SEQ ID NO: 3 and complements thereof.

In a further aspect, the present invention provides seed of any of the foregoing plants, various parts of plants which may express a desired sequence, and progeny of any of these transgenic plants as well.

Yet another aspect of the present invention is directed to methods for determining presence of sequence encoding □-tocopherol methyltransferase in a sample. Such methods include, without limitation, contacting the sample with a nucleic acid probe which hybridizes to a nucleic acid molecule having a sequence of any one of SEQ ID NO: 1-3 and determining whether the nucleic acid probe hybridizes to a nucleic acid in said sample. The present invention includes a method of screening for compounds capable of effecting the level of gamma-tocopherol methyltransferase expression comprising: (a) providing a cell with a nucleic acid sequence selected from the group consisting of SEQ ID NO: 1, SEQ ID NO: 2, SEQ ID NO: 3 and complements thereof operably linked to a heterologous nucleic acid sequence in manner where the complement of said heterologous nucleic acid sequence is expressed; (b) providing a test compound to said cell; and (c) determining the level of said complement of said heterologous nucleic acid sequence or a polypeptide encoded by said heterologous nucleic acid sequence. The present invention also includes a method of determining the presence of a nucleic acid sequence of at least 200 consecutive nucleotides in a sample comprising: (a) contacting the sample with a nucleic acid probe that hybridizes to a nucleic acid sequence having the sequence of SEQ ID NO: 1; and (b) determining whether the nucleic acid probe hybridizes to a nucleic acid molecule in said sample.

### BRIEF DESCRIPTION OF THE DRAWINGS

FIG. 1A is the sequence (SEQ ID NO: 4) of a clone obtained from the EcoRV library amplified with the E3-GSP1 and E3-GSP2 primers (RV2.1 clone). The sequence for Brassica napus GMT upstream sequence is in plain text (SEQ ID NO: 1) and the coding sequence is in bold (SEQ ID NO: 5).

FIG. 1B is the sequence (SEQ ID NO: 6) of the clone obtained from the PvuII digested library amplified with the E3-GSP1 and E3-GSP2 set of primers (pMON67501).

The sequence encoding Brassica napus GMT upstream sequence is in plain text (SEQ ID NO: 2) and the coding sequence is in bold (SEQ ID NO: 7).

FIG. 1C is the sequence (SEQ ID NO: 8) of the clone obtained from the StuI library amplified amplified with the E3-GSP1 and E3-GSP2 set of primers (pMON67502). The sequence encoding Brassica napus GMT upstream sequence is in plain text (SEQ ID NO: 3) and the coding sequence is in bold (SEQ ID NO: 9).

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, and other references cited in this application are herein incorporated by reference in their entirety as if each individual publication, patent, patent application, or other reference were specifically and individually indicated to be incorporated by reference.

In accordance with the present invention, three regulatory sequences upstream of the gene encoding gamma-tocopherol methyltransferase (GMT) in the genome of Brassica napus are isolated and sequenced. These regulatory sequences are identified herein as SEQ ID NOS: 1-3. Preferably, these regulatory sequences contain at least one transcription factor binding site, more preferably a binding site for a zinc finger transcription factor. Regulatory sequences can be used to regulate the expression of GMT thus enabling the increase or decrease of alpha-tocopherol levels in plant tissues. Thus, Applicants have identified nucleic acid sequences, vectors and methods that can be used to regulate expression, thereby allowing the manipulation of gene expression in plant tissues.

The following definitions and methods are provided to better define the present invention and to guide those of ordinary skill in the art in the practice of the present invention. The nomenclature for DNA bases as set forth at 37 CFR §1.822 is used. The standard one- and three-letter nomenclature for amino acid residues is used.

As used herein “isolated polynucleotide” means a polynucleotide that is free of one or both of the nucleotide sequences which flank the polynucleotide in the naturally-occurring genome of the organism from which the polynucleotide is derived. The term includes, for example, a polynucleotide or fragment thereof that is incorporated into a vector or expression cassette; into an autonomously replicating plasmid or virus; into the genomic DNA of a prokaryote or eukaryote; or that exists as a separate molecule independent of other polynucleotides. It also includes a recombinant polynucleotide that is part of a hybrid polynucleotide, for example, one encoding a polypeptide sequence.

As used herein “polynucleotide” and “oligonucleotide” are used interchangeably and refer to a polymeric (2 or more monomers) form of nucleotides of any length, either ribonucleotides or deoxyribonucleotides. Although nucleotides are usually joined by phosphodiester linkages, the term also includes polymeric nucleotides containing neutral amide backbone linkages composed of aminoethyl glycine units. This term refers only to the primary structure of the molecule. Thus, this term includes double- and single-stranded DNA and RNA. It also includes known types of modifications, for example, labels, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.), those containing pendant moieties, such as, for example, proteins (including for e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide. Polynucleotides include both sense and antisense strands.

“Native” refers to a naturally occurring (“wild-type”) nucleic acid sequence.

“Heterologous” sequence refers to a sequence that originates from a foreign DNA or species or, if from the same DNA, is modified from its original form.

The term “substantially purified”, as used herein, refers to a sequence separated from substantially all other molecules normally associated with it in its native state. More preferably, a substantially purified sequence is the predominant species present in a preparation. A substantially purified sequence may be greater than 60% free, preferably 75% free, more preferably 90% free from the other molecules (exclusive of solvent) present in the natural mixture. The term “substantially purified” is not intended to encompass sequences present in their native state.

A first nucleic acid sequence displays “substantial homology” to a reference nucleic acid sequence if, when optimally aligned (with appropriate nucleotide insertions or deletions totaling less than 20 percent of the reference sequence over the window of comparison) with the other nucleic acid (or its complementary strand), there is at least about 75% nucleotide sequence homology, preferably at least about 80% homology, more preferably at least about 85% homology, and most preferably at least about 90% homology over a comparison window of at least 20 nucleotide positions, preferably at least 50 nucleotide positions, more preferably at least 100 nucleotide positions, and most preferably over the entire length of the first nucleic acid. Optimal alignment of sequences for aligning a comparison window may be conducted by the local homology algorithm of Smith and Waterman, Adv. Appl. Math. 2:482, 1981; by the homology alignment algorithm of Needleman and Wunsch, J. Mol. Biol. 48:443, 1970; by the search for similarity method of Pearson and Lipman, Proc. Natl. Acad. Sci. USA 85:2444, 1988; preferably by computerized implementations of these algorithms (GAP, BESTFIT, FASTA, and TFASTA) in the Wisconsin Genetics Software Package Release 7.0, Genetics Computer Group, 575 Science Dr., Madison, Wis. Additional computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology. 12: 76-80 (1994); Birren, et al., Genome Analysis, 1: 543-559 (1997)). The BLASTN program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). In a preferred embodiment, the homology alignment algorithm of Smith and Waterman is implemented in the Wisconsin Genetics Software Package Release 7.0 as described previously. The reference nucleic acid may be a full-length molecule or a portion of a longer molecule. Alternatively, two nucleic acids have substantial identity if one hybridizes to the other under stringent conditions, as defined below.

A first nucleic acid sequence is “operably linked” with a second nucleic acid sequence when the sequences are so arranged that the first nucleic acid sequence affects the function of the second nucleic-acid sequence. Preferably, the two sequences are part of a single contiguous nucleic acid molecule and more preferably are adjacent. For example, a promoter is operably linked to a sequence if the promoter regulates or mediates transcription of the sequence in a cell.

A “recombinant” nucleic acid is made by an artificial combination of two otherwise separated segments of sequence, e.g., by chemical synthesis or by the manipulation of isolated segments of nucleic acids by genetic engineering techniques. Techniques for nucleic-acid manipulation are well-known in the art. See e.g., Molecular Cloning: A Laboratory Manual, 2nd ed., vol. 1-3, ed. Sambrook et al., Cold Spring Harbor Laboratory Press, Cold Spring Harbor, N.Y., 1989 (“Sambrook et al., 1989”); Current Protocols in Molecular Biology, ed. Ausubel et al., Greene Publishing and Wiley-Interscience, New York, 1992 (with periodic updates) (Ausubel et al., 1992). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

A “synthetic nucleic acid sequence” can be designed and chemically synthesized for enhanced expression in particular host cells and for the purposes of cloning into appropriate vectors. Host cells often display a preferred pattern of codon usage (Murray et al., 1989 Nucleic Acids Res. 2: 477-98). Synthetic DNAs designed to enhance expression in a particular host should therefore reflect the pattern of codon usage in the host cell. Computer programs are available for these purposes including but not limited to the “BestFit” or “Gap” programs of the Sequence Analysis Software Package, Genetics Computer Group, Inc., University of Wisconsin Biotechnology Center, Madison, Wis. 53711.

“Amplification” of nucleic acids or “nucleic acid reproduction” refers to the production of additional copies of a nucleic acid sequence and is carried out using polymerase chain reaction (PCR) technologies. A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. In PCR, a primer refers to a short oligonucleotide of defined sequence that is annealed to a DNA template to initiate the polymerase chain reaction.

“Transformed”, “transfected”, or “transgenic” refers to a cell, tissue, organ, or organism into which has been introduced a foreign nucleic acid, such as a recombinant vector. Preferably, the introduced nucleic acid is integrated into the genomic DNA of the recipient cell, tissue, organ or organism such that the introduced nucleic acid is inherited by subsequent progeny. A “transgenic” or “transformed” cell or organism also includes progeny of the cell or organism and progeny produced from a breeding program employing such a “transgenic” plant as a parent in a cross and exhibiting an altered phenotype resulting from the presence of a recombinant construct or vector.

“Expression” of a gene refers to the transcription of a gene to produce the corresponding mRNA and may further include translation of this mRNA to produce the corresponding gene product, i.e., a peptide, polypeptide, or protein. Gene expression is controlled or modulated by regulatory elements including 5′ regulatory elements such as regulatory sequences.

“Genetic component” refers to any nucleic acid sequence or genetic element that may also be a component or part of an expression vector. Examples of genetic components include, but are not limited to promoter regions, 5′ untranslated leaders, introns, genes, 3′ untranslated regions, and other regulatory sequences or sequences that affect transcription or translation of one or more nucleic acid sequences.

The terms “recombinant DNA construct”, “recombinant vector”, “expression vector” or “expression cassette” refer to any agent such as a plasmid, cosmid, virus, BAC (bacterial artificial chromosome), autonomously replicating sequence, phage, or linear or circular single-stranded or double-stranded DNA or RNA nucleotide sequence, derived from any DNA, capable of genomic integration or autonomous replication, comprising a DNA molecule in which one or more DNA sequences have been linked in a functionally operative manner.

“Complementary” refers to the natural association of nucleic acid sequences by base-pairing (A-G-T pairs with the complementary sequence T-C-A). Complementarity between two single-stranded molecules may be partial, if only some of the nucleic acids pair are complementary; or complete, if all bases pair are complementary. The degree of complementarity affects the efficiency and strength of hybridization and amplification reactions.

“Homology” refers to the level of similarity between nucleic acid or amino acid sequences in terms of percent nucleotide or amino acid positional identity, respectively, i.e., sequence similarity or identity. Homology also refers to the concept of similar functional properties among different nucleic acids or proteins.

“Promoter” refers to a nucleic acid sequence located upstream or 5′ to a translational start codon of an open reading frame (or protein-coding region) of a gene and that is involved in recognition and binding of RNA polymerase II and other proteins (trans-acting transcription factors) to initiate transcription. A “plant promoter” is a native or non-native promoter that is functional in plant cells. Constitutive regulatory sequences are functional in most or all tissues of a plant throughout plant development. Tissue-, organ- or cell-specific regulatory sequences are expressed only or predominantly in a particular tissue, organ, or cell type, respectively. Rather than being expressed “specifically” in a given tissue, organ, or cell type, a promoter may display “enhanced” expression, i.e., a higher level of expression, in one part (e.g., cell type, tissue, or organ) of the plant compared to other parts of the plant. Temporally regulated regulatory sequences are functional only or predominantly during certain periods of plant development or at certain times of day, as in the case of genes associated with circadian rhythm, for example. Inducible regulatory sequences selectively express an operably linked DNA sequence in response to the presence of an endogenous or exogenous stimulus, for example by chemical compounds (chemical inducers) or in response to environmental, hormonal, chemical, and/or developmental signals. Inducible or regulated regulatory sequences include, for example, regulatory sequences regulated by light, heat, stress, flooding or drought, phytohormones, wounding, or chemicals such as ethanol, jasmonate, salicylic acid, or safeners.

“GMT” is gamma-tocopherol methyltransferase. A GMT enzyme catalyzes the methylation of gamma-tocopherol to form alpha-tocopherol, the final step of alpha tocopherol biosynthesis.

“ZFP” is zinc finger protein. A zinc finger is one of the major structural motifs involved in eukaryotic protein-nucleic acid interaction.

Regulatory sequences of the present invention are useful for regulating expression of a target polypeptide, preferably GMT in plant tissues. The availability of suitable regulatory sequences that regulate transcription of operably linked sequences in selected target tissues of interest is important since it may not be desirable to have expression of a sequence in every tissue, but only in certain tissues. Regulatory sequences of the present invention are capable of regulating expression of operably linked DNA sequences in plant tissues and have utility for regulating transcription of any target sequence, preferably sequences encoding for GMT, the enzyme which catalyzes the methylation of gamma-tocopherol to form alpha-tocopherol.

Applicants have isolated the sequences upsteam of the ATG start codon of sequences encoding GMT in Brassica napus and disclosed three different sequences. These data suggest that there is more than one copy of this sequence in the genome. Southern data further supports the fact that there are four distinct GMT sequences present in the Brassica napus genome.

Regulatory sequences of the present invention can be used as plant promoters. A plant promoter can be used as a 5′ regulatory sequence to regulate expression of a particular nucleotide sequence(s). One example of a promoter is a plant RNA polymerase II promoter. Plant RNA polymerase II promoter, like those of other higher eukaryotes, has complex structures and is comprised of several distinct elements. One such element is the TATA box or Goldberg-Hogness box, which is required for correct expression of eukaryotic sequences in vitro and accurate, efficient initiation of transcription in vivo. The TATA box is typically positioned at approximately −25 to −35, that is, at 25 to 35 basepairs (bp) upstream (5′) of the transcription initiation site, or cap site, which is defined as position +1 (Breathnach and Chambon, Ann. Rev. Biochem. 50:349-383, 1981; Messing et al., In: Genetic Engineering of Plants, Kosuge et al., eds., pp. 211-227, 1983). Another common element, the CCAAT box, is located between −70 and −100 bp. In plants, the CCAAT box can have a different consensus sequence than the functionally analogous sequence of mammalian regulatory sequences (the plant analogue has been termed the “AGGA box” to differentiate it from its animal counterpart; Messing et al., In: Genetic Engineering of Plants, Kosuge et al., eds., pp. 211-227, 1983). In addition, many regulatory sequences include additional upstream activating sequences or enhancers (Benoist and Chambon, Nature 290:304-310, 1981; Gruss et al., Proc. Nat. Acad. Sci. USA 78:943-947, 1981; and Khoury and Gruss, Cell 27:313-314, 1983) extending from around −100 bp to −1,000 bp or more upstream of the transcription initiation site.

When fused to heterologous DNA sequences, regulatory sequences of the present invention preferably cause the fused sequence to be transcribed in a manner that is similar to that of the sequence that the regulatory sequence is normally associated with. Additionally, one skilled in the art can add heterologous regulatory sequences to the 5′ upstream region of the regulatory sequences of the present invention e.g., an inactive, truncated promoter, e.g., a promoter including only the core TATA and, sometimes, the CCAAT elements (Fluhr et al., Science 232:1106-1112, 1986; Strittmatter and Chua, Proc. Nat. Acad. Sci. USA 84:8986-8990, 1987; Aryan et al., Mol. Gen. Genet. 225:65-71, 1991).

To identify the nucleic acid sequences of the present invention from a database or collection of cDNA sequences, the first step involves constructing cDNA libraries from specific plant tissue targets of interest. Briefly, the cDNA libraries are first constructed from these tissues that are harvested at a particular developmental stage, or under particular environmental conditions. By identifying differentially expressed genes in plant tissues at different developmental stages, or under different conditions, the corresponding regulatory sequences of those genes can be identified and isolated. Transcript imaging enables the identification of tissue-preferred sequences based on specific imaging of nucleic acid sequences from a cDNA library. By transcript imaging as used herein is meant an analysis that compares the abundance of expressed genes in one or more libraries. The clones contained within a cDNA library are sequenced and the sequences compared with sequences from publicly available databases. Computer-based methods allow the researcher to provide queries that compare sequences from multiple libraries. The process enables quick identification of clones of interest compared with conventional hybridization subtraction methods known to those of skill in the art.

Using conventional methodologies, cDNA libraries can be constructed from the mRNA (messenger RNA) of a given tissue or organism using poly dT primers and reverse transcriptase (Efstratiadis, et al., Cell 7:279, 1976; Higuchi, et al., Proc. Natl. Acad. Sci. USA 73:3146, 1976; Maniatis, et al., Cell 8:163, 1976; Land et al., Nucleic Acids Res. 9:2251, 1981; Okayama, et al., Mol. Cell. Biol. 2:161, 1982; Gubler, et al., Gene 25:263, 1983).

Several methods can be employed to obtain full-length cDNA constructs. For example, terminal transferase can be used to add homopolymeric tails of dC residues to the free 3′ hydroxyl groups (Land, et al., Nucleic Acids Res. 9:2251, 1981). This tail can then be hybridized by a poly dG oligo that can act as a primer for the synthesis of full length second strand cDNA. Okayama and Berg, reported a method for obtaining full length cDNA constructs (Mol. Cell Biol. 2:161 (1982). This method has been simplified by using synthetic primer-adapters that have both homopolymeric tails for priming the synthesis of the first and second strands and restriction sites for cloning into plasmids (Coleclough, et al., Gene 34:305, 1985) and bacteriophage vectors (Krawinkel, et al., Nucleic Acids Res. 14:1913, 1986; and Han, et al., Nucleic Acids Res. 15:6304, 1987).

These strategies can be coupled with additional strategies for isolating rare mRNA populations. For example, a typical mammalian cell contains between 10,000 and 30,000 different mRNA sequences, (Davidson, Gene Activity in Early Development, 2nd ed., Academic Press, New York, 1976). The number of clones required to achieve a given probability that a low-abundance mRNA will be present in a cDNA library is N=(ln(l−P))/(ln(l−l/n)) where N is the number of clones required, P is the probability desired, and l/n is the fractional proportion of the total mRNA that is represented by a single rare mRNA (Sambrook, et al., 1989).

One method to enrich preparations of mRNA for sequences of interest is to fractionate by size. One such method is to fractionate by electrophoresis through an agarose gel (Pennica, et al., Nature 301:214, 1983). Another method employs sucrose gradient centriflugation in the presence of an agent, such as methylmercuric hydroxide, that denatures secondary structure in RNA (Schweinfest, et al., Proc. Natl. Acad. Sci. USA 79:4997-5000, 1982).

A frequently adopted method is to construct equalized or normalized cDNA libraries (Ko, Nucleic Acids Res. 18:5705, 1990; Patanjali, S. R. et al., Proc. Natl. Acad. Sci. USA 88:1943, 1991). Typically, the cDNA population is normalized by subtractive hybridization. Schmid, et al., J. Neurochem. 48:307, 1987; Fargnoli, et al., Anal. Biochem. 187:364, 1990; Travis, et al., Proc. Natl. Acad. Sci USA 85:1696, 1988; Kato, Eur. J. Neurosci. 2:704, 1990; and Schweinfest, et al., Genet. Anal. Tech. Appl. 7:64, 1990). Subtraction represents another method for reducing the population of certain sequences in the cDNA library, (Swaroop, et al., Nucleic Acids Res. 19:1954, 1991). Normalized libraries can be constructed using the Soares procedure (Soares et al., Proc. Natl. Acad. Sci. USA 91:9228, 1994). This approach is designed to reduce the initial 10,000-fold variation in individual cDNA frequencies to achieve abundances within one order of magnitude while maintaining the overall sequence complexity of the library. In the normalization process, the prevalence of high-abundance cDNA clones decreases dramatically, clones with mid-level abundance are relatively unaffected, and clones for rare transcripts are effectively increased in abundance.

Any type of plant tissue can be used as a target tissue for the identification of regulatory sequences. For example without limitation, plant tissue from Brassica napus is used to identify the regulatory sequences as identified herein as SEQ ID NOS: 1-3. Brassica napus cDNA libraries can be constructed from several different plant developmental stages. Background or non-target libraries can include but are not limited to libraries such as leaf, root, embryo, callus, shoot, seedling, endosperm, culm, ear, and silks.

Differential hybridization techniques as described are well known to those of skill in the art and can also be used to isolate a desired class of sequences. By classes of sequences as used herein is meant sequences that can be grouped based on a common identifier including but not limited to sequences isolated from a common target plant, a common library, or a common plant tissue type. In a preferred embodiment, sequences of interest are identified based on sequence analyses and querying of a collection of diverse cDNA sequences from libraries of different tissue types.

A number of methods used to assess gene expression are based on measuring the mRNA level in an organ, tissue, or cell sample. Typical methods include but are not limited to RNA blots, ribonuclease protection assays and RT-PCR. In another preferred embodiment, a high-throughput method is used whereby regulatory sequences are identified from a transcript profiling approach. The development of cDNA microarray technology enables the systematic monitoring of gene expression profiles for thousands of genes (Schena et al, Science, 270: 467, 1995). This DNA chip-based technology arrays thousands of cDNA sequences on a support surface. These arrays are simultaneously hybridized to multiple labeled cDNA probes prepared from RNA samples of different cell or tissue types, allowing direct comparative analysis of expression. This technology was first demonstrated by analyzing 48 Arabidopsis genes for differential expression in roots and shoots (Schena et al, Science, 270:467, 1995). More recently, the expression profiles of over 1400 genes were monitored using cDNA microarrays (Ruan et al, The Plant Journal 15:821, 1998). Microarrays provide a high-throughput, quantitative and reproducible method to analyze gene expression and characterize gene function. The transcript profiling approach using microarrays thus provides another valuable tool for the isolation of regulatory sequences such as regulatory sequences associated with those genes.

The present invention uses high throughput sequence analyses to form the foundation of rapid computer-based identification of sequences of interest. Those of skill in the art are aware of the resources available for sequence analyses. Sequence comparisons can be done by determining the similarity of the test or query sequence with sequences in publicly available or proprietary databases (“similarity analysis”) or by searching for certain motifs (“intrinsic sequence analysis”) (e.g. cis elements) (Coulson, Trends in Biotechnology, 12:76, 1994; Birren, et al., Genome Analysis, 1:543, 1997).

In a preferred embodiment, the nucleic acid sequences of the regulatory elements of the present invention are isolated from a Brassicacae, preferably Brassica napus, using a genome-walking approach (Universal GenomeWalker™ Kit, CLONTECH Laboratories, Inc., Palo Alto, Calif.). Briefly, the purified genomic DNA is subjected to a restriction enzyme digest that produces genomic DNA fragments with ends that are ligated with GenomeWalker™ adaptors. GenomeWalker™ primers are used along with gene specific primers in two consecutive PCR reactions (primary and nested PCR reactions) to produce PCR products containing the 5′ regulatory sequences that are subsequently cloned and sequenced.

The present invention includes, without limitation, the regulatory sequences of SEQ ID NOS: 1-3 or the complement thereof and any nucleic acid hybridizing under stringent conditions to any one of the sequences of SEQ ID NOS: 1-3. Nucleic acid fragments can also be obtained by other techniques such as by directly synthesizing the fragment by chemical means, as is commonly practiced by using an automated oligonucleotide synthesizer. Fragments can also be obtained by application of nucleic acid reproduction technology, such as the PCR (polymerase chain reaction) technology by recombinant DNA techniques generally known to those of skill in the art of molecular biology. PCR is a rapid and simple method for specifically amplifying a target DNA sequence in an exponential manner. See Saiki, et al. Science 239:487-4391 (1988); U.S. Pat. Nos. 4,683,195 and 4,683,202. Briefly, the method as now commonly practiced utilizes a pair of primers that have nucleotide sequences complementary to the DNA which flanks the target sequence. The primers are mixed with a solution containing the target DNA (the template), a DNA polymerase and dNTPS for all four deoxynucleotides (adenosine (A), tyrosine (T), cytosine (C) and guanine(G)). The mix is then heated to a temperature sufficient to separate the two complementary strands of DNA. The mix is next cooled to a temperature sufficient to allow the primers to specifically anneal to sequences flanking the sequence or sequences of interest. The temperature of the reaction mixture is then set to the optimum for the thermophilic DNA polymerase to allow DNA synthesis (extension) to proceed. The temperature regimen is then repeated to constitute each amplification cycle. Thus, PCR consists of multiple cycles of DNA melting, annealing and extension. Twenty replication cycles can yield up to a million-fold amplification of the target DNA sequence. In some applications a single primer sequence functions to prime at both ends of the target, but this only works efficiently if the primer is not too long in length. In some applications several pairs of primers are employed in a process commonly known as multiplex PCR.

A fragment of a nucleic acid as used herein is a portion of the nucleic acid that is less than full-length. For example, for the present invention any length of nucleotide sequence that is less than the disclosed nucleotide sequences of SEQ ID NOS: 1-3 is considered to be a fragment. A fragment can also comprise at least a minimum length capable of hybridizing specifically with a native nucleic acid under stringent hybridization conditions as defined above. The length of such a minimal fragment is preferably at least 15 consecutive nucleotides, more preferably at least 20 consecutive nucleotides, and even more preferably at least 30 consecutive nucleotides of a native nucleic acid sequence. In a preferred aspect of the present invention, a fragment consists of at least 50 consecutive nucleotides or at least 75 consecutive nucleotides. In a highly preferred aspect, a fragment consists of at least 100 consecutive nucleotides or at least 150 consecutive nucleotides. In a more highly preferred aspect, a fragment consists of at least 200 consecutive nucleotides or at least 250 consecutive nucleotides.

Fragment nucleic acid molecules may consist of significant portion(s) of, or indeed most of, the nucleic acid molecules of the invention, such as those specifically disclosed. Alternatively, the fragments may comprise smaller oligonucleotides with at least a minimum length from about 15 consecutive to about 400 consecutive nucleotide residues and more preferably, about 15 consecutive to about 30 consecutive nucleotide residues, or about 50 consecutive to about 100 consecutive nucleotide residues, or about 100 consecutive to about 200 consecutive nucleotide residues, or about 200 consecutive to about 400 consecutive nucleotide residues, or about 275 consecutive to about 350 consecutive nucleotide residues capable of hybridizing specifically with a native nucleic acid under stringent hybridization conditions.

A fragment of one or more of the nucleic acid molecules of the invention may be a probe and specifically a PCR probe. A PCR probe is a nucleic acid molecule capable of initiating a polymerase activity while in a double-stranded structure with another nucleic acid. Various methods for determining the structure of PCR probes and PCR techniques exist in the art. Computer generated searches using programs such as Primer3 (www-genome.wi.mit.edu/cgi-bin/primer/primer3.cgi), STSPipeline (www-genome.wi.mit.edu/cgi-bin/www-STS_Pipeline), or GeneUp (Pesole et al., BioTechniques 25:112-123 (1998)), for example, can be used to identify potential PCR primers.

Further, the nucleotide sequences of the regulatory sequences disclosed herein can be modified. Those skilled in the art can create DNA sequences that have variations in the nucleotide sequence. The nucleotide sequences as shown in SEQ ID NOS: 1-3 may be modified or altered to enhance their control characteristics. One preferred method of alteration of a nucleic acid sequence is to use PCR to modify selected nucleotides or regions of sequences. These methods are known to those of skill in the art. Sequences can be modified, for example by insertion, deletion or replacement of template sequences in a PCR-based DNA modification approach. “Variant” DNA sequences are DNA sequences containing changes in which one or more nucleotides of a native sequence is deleted, added, and/or substituted, preferably while substantially maintaining regulatory sequence function. In the case of a regulatory sequence fragment, “variant” DNA can include changes affecting the transcription of the polypeptide to which it is operably linked. Variant DNA sequences can be produced, for example, by standard DNA mutagenesis techniques or by chemically synthesizing the variant DNA molecule or a portion thereof.

Preferably, one or more of the three identified regulatory sequences of SEQ ID NOS: 1-3 and complements thereof and fragments of either contain promoters, specifically, inducible promoters, constitutive promoters, developmentally regulated promoters or tissue specific promoters, and preferably, seed-specific promoters. The isolated regulatory sequences of the present invention can be incorporated into recombinant nucleic acid constructs, typically DNA constructs, capable of introduction into and replication in a host cell. The regulatory sequences preferably contain at least one transcription factor binding site and are capable of transcribing operably linked DNA sequences in plant tissues. The nucleic acid sequences of the present invention can be operably linked to any nucleic acid sequence of interest such as a nucleic acid that confers a desirable characteristic associated with plant morphology, physiology, growth and development, yield, nutritional enhancement, disease or pest resistance, or environmental or chemical tolerance in an expression vector. These genetic components, such as marker genes or agronomic sequences of interest, can function in the identification of a transformed plant cell or plant, or produce a product of agronomic utility. In a preferred embodiment, one genetic component produces a product that serves as a selection device and functions in a regenerable plant tissue to produce a compound that would confer upon the plant tissue resistance to an otherwise toxic compound. Genes of interest for use as a selectable, screenable, or scorable marker include but are not limited to GUS (coding sequence for beta-glucuronidase), GFP (coding sequence for green fluorescent protein), LUX (coding gene for luciferase), antibiotic resistance marker genes, or herbicide tolerance genes. Examples of transposons and associated antibiotic resistance genes include the transposons Tns (bla), Tn5 (nptII), Tn7 (dhfr), penicillins, kanamycin (and neomycin, G418, bleomycin); methotrexate (and trimethoprim); chloramphenicol; kanamycin and tetracycline.

Characteristics useful for selectable markers in plants have been outlined in a report on the use of microorganisms (Advisory Committee on Novel Foods and Processes, July 1994). These include stringent selection with minimum number of nontransformed tissues, large numbers of independent transformation events with no significant interference with the regeneration, application to a large number of species, and availability of an assay to score the tissues for presence of the marker.

A number of selectable marker genes are known in the art and several antibiotic resistance markers satisfy these criteria, including those resistant to kanamycin (nptII), hygromycin B (aph IV) and gentamycin (aac3 and aacC4). Useful dominant selectable marker genes include genes encoding antibiotic resistance genes (e.g., resistance to hygromycin, kanamycin, bleomycin, G418, streptomycin or spectinomycin); and herbicide resistance genes (e.g., phosphinothricin acetyltransferase). A useful strategy for selection of transformants for herbicide resistance is described, e.g., in Vasil, Cell Culture and Somatic Cell Genetics of Plants. Vols. I-III, laboratory Procedures and Their Applications Academic Press, New York, 1984. Particularly preferred selectable marker genes for use in the present invention would include genes that confer resistance to compounds such as antibiotics like kanamycin, and herbicides like glyphosate (Della-Cioppa et al., Bio/Technology 5(6), 1987, U.S. Pat. No. 5,463,175, U.S. Pat. No. 5,633,435). Other selection devices can also be implemented and would still fall within the scope of the present invention.

Plant expression vectors can also include additional elements such as RNA processing signals, e.g., introns, which may be positioned upstream or downstream of a polypeptide-encoding sequence in the transgene. In addition, the expression vectors may include additional regulatory sequences from the 3′-untranslated region of plant genes (Thornburg et al., Proc. Natl. Acad. Sci. USA 84:744 (1987); An et al., Plant Cell 1:115 (1989)), e.g., a 3′ terminator region to increase mRNA stability of the mRNA, such as the PI-II terminator region of potato or the octopine or nopaline synthase 3′ terminator regions. 5′ non-translated regions of a mRNA can play an important role in translation initiation and can also be a genetic component in a plant expression vector. For example, non-translated 5′ leader sequences derived from heat shock protein genes have been demonstrated to enhance gene expression in plants (see, for example U.S. Pat. No. 5,362,865). These additional upstream and downstream regulatory sequences may be derived from a DNA that is native or heterologous with respect to the other elements present on the expression vector.

In a preferred aspect, the regulatory sequences contain at least one transcription factor binding site. Preferably, these regions are binding sites for zinc-finger transcription factors. Transcription factors that function to direct the localization of enzymes to specific DNA addresses are dependent on the availability of sequence-specific DNA-binding domains. Of the DNA binding domains that have been studied, the modular zinc finger DNA binding domains of the Cys₂-His₂ class have shown the most promise for the development of a universal system for gene regulation (Berg et al., 1996. Science 271:1081-1085; Berg, J. M. 1997. Nature Biotechnology:323; Choo et al., 1997 Journal of Molecular Biology 273: 525-532; Greisman et al., 1997 Science 275:657-661). Zinc-finger proteins are known as a class of diverse eukaryotic transcription factors that utilize zinc-containing DNA-binding domains and are important regulators of development. See McKnight, S. L. and K. R. Yamamoto, eds. (1992) Transcriptional Regulation, Cold Spring Harbor Laboratory Press, Cold Spring Harbor, N.Y., Vol. 1, p. 580. Zinc-finger proteins exert a regulatory function by mediating the transcription of other sequences. Identification of these binding regions enables the design and production of new zinc finger protein transcription factors to bind to one, some or all of the zinc finger binding domains present in the regulatory sequences. Recent progress in the design and selection of novel zinc finger binding proteins with desired DNA binding specificities now allows construction of tailor-made DNA-binding proteins that specifically recognize predetermined DNA sequences. By modifying those portions of a zinc finger binding proteins that interact with DNA, new zinc finger binding proteins can be created capable of recognizing DNA sequences in virtually any nucleic acid sequence whose sequence is known. (Liu et al, 1997 Proc. Natl. Acad. Sci. 94:5525-5530; Pavletich et al., 1991 Science 252:809-817; Rebar et al., 1994 Science 263:671-673; Wang et al., 1999 Proceedings of the National Academy of Sciences 96:9568-9573). Multiple zinc finger binding proteins can be linked together to recognize longer stretches of DNA (Beerli et al., 1998 Proc. Natl. Acad. Sci. 95:14628-14633; Kim et al., 1997 Proc. Natl. Acad. Sc. 94:3616-3620; Kim et al., 1998 Proc. Natl. Acad. Sci. 95:2812-2817).

Zinc finger protein transcription factors have two distinct elements or domains: the DNA recognition domain that directs the zinc finger protein transcription factor to the proper chromosomal location by recognizing a specific DNA sequence and a functional domain which causes the zinc finger protein transcription factor to control or regulate the nucleic acid sequence in a desired manner. An activation domain causes a target sequence to be turned on and alternatively a repression domain causes the sequence to be turned off (Beerli et al., 2000 Proc. Natl. Acad. Sci. 97:1495-1500; Kim et al., 1997 Journal of Biological Chemistry 272:29795-29800). By coupling the zinc finger binding protein DNA recognition domain designed to bind to the region upstream of any given target sequence to a specific functional domain it is possible to cause zinc finger binding protein transcription factors to control or regulate the expression of a target sequence in a desired manner (Kang et al., 2000 Journal of Biological Chemistry 275:8742-8748).

Zinc finger proteins have been successfully used in plants to direct the expression of latent transgenes (Guyer et al., 1998. Genetics 149:633-639) using the C1 activation domain from maize (Goffet al., 1998 Genes and Development 5:298-309). In a preferred aspect, transgenic plants express zinc finger protein transcription factors designed to bind to the endogenous sequences located upstream of the gamma-tocopherol methyl transferase (GMT) coding regions (described in this patent) in Brasssica napus. This protein will be coupled to an activation domain that is functional in plants. Transgenic expression of these engineered zinc finger transcription factors will lead to the activation of the GMT gene and expression of the GMT protein in those tissues where the zinc finger protein is present.

Binding of a transcription factor to these regulatory sequences will allow for the regulation of expression of a polypeptide operably linked to the regulatory sequence. In a preferred embodiment, transcription factors are specifically designed to recognize and bind one or more of the binding sites of the regulatory sequences thereby activating transcription of the adjacent GMT coding region. The seed profile of Arabidopsis and major oilseed crops is >95% gamma-tocopherol. Transgenic overexpression of the GMT protein in the seeds of Arabidopsis was shown to result in a conversion of the tocopherol content of the seeds to >95% alpha-tocopherol (Shintani et al., 1998, Science 282:2098-2100.). Gamma-tocopherol methyl transferase (GMT) is the key enzymatic activity involved in determining the tocopherol composition of seeds. Preferably, transgenic expression of a ZFP transcription factor with the properties of those described above under the control of the napin promoter in Brassica napus plants would cause the expression of GMT in the seed which would have the effect of catalyzing the conversion of the seed pool of gamma-tocopherol to alpha-tocopherol. This would increase the alpha-tocopherol content and hence the vitamin E activity of canola seed and seed oil derived from transgenic plants expressing the ZFP transcription factor.

Preferably, plants transfected with vectors containing nucleic acid sequences encoding these zinc finger transcription factors capable of binding to the regulatory sequence of any one of SEQ ID NOS: 1-3 or complements thereof or fragments of either will result in the increased control of the expression of GMT protein in the plant tissue. The use of seed-specific promoter in such vectors will enable the expression of a polypeptide, preferably GMT, in plant seed. Preferably, the expression of GMT will catalyze the conversion of gamma-tocopherol to alpha-tocopherol thereby resulting in increased levels of alpha-tocopherol in the plant seed.

Another aspect of the present invention is directed to a vector comprising a nucleic acid sequence encoding a transcription factor which binds to regulatory sequences having the sequence of any one of SEQ ID NO: 1-3 or complements thereof or fragments of either operably linked to a polypeptide of interest, whereby expression of the transcription factor regulates expression of the polypeptide of interest. Preferably, the vector contains nucleic acid sequences encoding for a zinc finger transcription factor which binds to one or more binding sites of the any one of SEQ ID NOS: 1-3 or complements thereof or fragments of either. In another preferred embodiment, the polypeptide of interest comprises GMT.

In a preferred embodiment, regulation of the expression of a polypeptide in a cell includes transfecting the cell, preferably, a plant cell with a vector comprising a nucleic acid molecule encoding a transcription factor which binds to SEQ ID NO: 1 or complements thereof or fragments of either, whereby expression of the transcription factor regulates expression of the polypeptide in the cell. Preferably, the transcription factor is a zinc finger transcription factor and the polypeptide is GMT. In another preferred embodiment, the transcription factor binds to SEQ ID NO: 2 or complements thereof or fragments of either. In yet another preferred embodiment, the transcription factor binds to SEQ ID NO: 3 or complements thereof or fragments of either.

Regulatory sequences of the present invention are preferably used to control nucleic acid sequence expression in plant cells. The disclosed regulatory sequences are genetic components that can be part of vectors used in plant transformation. Sequences of the present invention can be used with any suitable plant transformation plasmid or vector, preferably those containing a selectable or screenable marker and associated regulatory elements, as described herein, along with one or more nucleic acids expressed in a manner sufficient to confer a particular desirable trait. Examples of suitable structural genes of agronomic interest envisioned by the present invention would include but are not limited to one or more genes for insect tolerance, such as a B.t., pest tolerance such as genes for fungal disease control, herbicide tolerance such as genes conferring glyphosate tolerance, and genes for quality improvements such as yield, nutritional enhancements, environmental or stress tolerances, or any desirable changes in plant physiology, growth, development, morphology or plant product(s). In a preferred embodiment, the particular desirable trait in the increased expression of GMT in plant tissue.

An aspect of the invention is directed to host cells comprising at least one of the above mentioned vectors containing the transcription factors which bind to the regulatory sequence of any one of SEQ ID NOS: 1-3 or complements thereof or fragments of either. For the practice of the present invention, conventional compositions and methods for preparing and using vectors and host cells are employed, as discussed, inter alia, in Sambrook et al., 1989. In a preferred embodiment, the host cell is a plant cell. A number of vectors suitable for stable transfection of plant cells or for the establishment of transgenic plants have been described in, e.g., Pouwels et al., Cloning Vectors: A Laboratory Manual, 1985, supp. 1987); Weissbach and Weissbach, Methods for Plant Molecular Biology, Academic Press, 1989; Gelvin et al., Plant Molecular Biology Manual, Kluwer Academic Publishers, 1990; and R. R. D. Croy, Plant Molecular Biology LabFax, BIOS Scientific Publishers, 1993. Plant expression vectors can include, for example, one or more cloned plant nucleotide sequences under the transcriptional control of 5′ and 3′ regulatory sequences. They can also include a selectable marker as described herein to select for host cells containing the expression vector. Such plant expression vectors may also contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and a polyadenylation signal. In a preferred embodiment, the host cell is a plant cell and the plant expression vector comprises a nucleic acid encoding a transcription factor which binds to a region as disclosed in SEQ ID NOS: 1-3 or complements thereof or fragments of either. Other regulatory sequences envisioned as genetic components in an expression vector include but is not limited to non-translated leader sequence that can be coupled with the promoter.

Another aspect of the present invention is the provision of transgenic plants produced using the nucleic acid constructs and expression vectors described herein. Methods for specifically transforming dicots primarily use Agrobacterium tumefaciens. For example, transgenic plants reported include, but are not limited to, cotton (U.S. Pat. No. 5,004,863; U.S. Pat. No. 5,159,135; U.S. Pat. No. 5,518,908, WO 97/43430), soybean (U.S. Pat. No. 5,569,834; U.S. Pat. No. 5,416,011; McCabe et al., Bio/Technology, 6:923, 1988; Christou et al., Plant Physiol., 87:671, 1988); Brassica (U.S. Pat. No. 5,463,174), tobacco (U.S. Pat. No. 5,861,277), Arabidopsis (U.S. Pat. No. 6,100,450) and peanut (Cheng et al., Plant Cell Rep., 15: 653, 1996).

Similar methods have been reported in the transformation of monocots. Transformation and plant regeneration using these methods have been described for a number of crops including but not limited to asparagus (Asparagus officinalis; Bytebier et al., Proc. Natl. Acad. Sci. U.S.A., 84: 5345, 1987); barley (Hordeum vulgarae; Wan and Lemaux, Plant Physiol., 104: 37, 1994); maize (Zea mays; Rhodes, C. A., et al., Science, 240: 204, 1988; Gordon-Kamm, et al., Plant Cell, 2: 603, 1990; Fromm, et al., Bio/Technology, 8: 833, 1990; Koziel, et al., Bio/Technology, 11: 194, 1993); oats (Avena sativa; Somers, et al., Bio/Technology, 10: 1589, 1992); orchardgrass (Dactylis glomerata; Horn, et al., Plant Cell Rep., 7: 469, 1988); rice (Oryza sativa, including indica and japonica varieties, Toriyama, et al., Bio/Technology, 6: 10, 1988; Zhang, et al., Plant Cell Rep., 7: 379, 1988; Luo and Wu, Plant Mol. Biol. Rep., 6: 165, 1988; Zhang and Wu, Theor. Appl. Genet., 76: 835, 1988; Christou, et al., Bio/Technology, 9: 957, 1991); sorghum (Sorghum bicolor; Casas, A.M., et al., Proc. Natl. Acad. Sci. U.S.A., 90: 11212, 1993); sugar cane (Saccharum spp.; Bower and Birch, Plant J., 2: 409, 1992); tall fescue (Festuca arundinacea; Wang, Z. Y. et al., Bio/Technology, 10: 691, 1992); turfgrass (Agrostis palustris; Zhong et al., Plant Cell Rep., 13: 1, 1993); wheat (Triticum aestivum; Vasil et al., Bio/Technology, 10: 667, 1992; Weeks T., et al., Plant Physiol., 102: 1077, 1993; Becker, et al., Plant, J. 5: 299, 1994), and alfalfa (Masoud, S. A., et al., Transgen. Res., 5: 313, 1996). It is apparent to those of skill in the art that a number of transformation methodologies can be used and modified for production of stable transgenic plants from any number of target crops of interest.

Transformed plants are analyzed for the presence of the nucleic acid sequences of interest and the expression level and/or profile conferred by the sequences of the present invention. Those of skill in the art are aware of the numerous methods available for the analysis of transformed plants. A variety of methods are used to assess sequence expression and determine if the introduced sequence(s) is integrated, functioning properly, and inherited as expected. For the present invention the regulatory sequences can be evaluated by determining the expression levels of sequences to which the regulatory sequences are operatively linked. A preliminary assessment of promoter function can be determined by a transient assay method using reporter genes, but a more definitive promoter assessment can be determined from the analysis of stable plants. Methods for plant analysis include but are not limited to Southern blots or northern blots, PCR-based approaches, biochemical analyses, phenotypic screening methods, field evaluations, and immunodiagnostic assays.

It should be noted that GMT may be found in the various parts of such transgenic plants encompassed herein. While the regulatory sequences contemplated in the present invention function preferentially in seed tissues, expression in other plant parts is also within the scope of the present invention, depending upon the specificity of the particular sequence. In one aspect, regulatory sequences functional in plant plastids are used to drive expression of the recombinant constructs disclosed herein in plastids present in tissues and organs other than seeds. For example, expression of a sequence, preferably GMT, can be expected in fruits, as well as vegetable parts of plants other than seeds. Vegetable parts of plants include, for example, pollen, inflorescences, terminal buds, lateral buds, stems, leaves, tubers, and roots. Thus, the present invention also encompasses these and other parts of the plants disclosed herein that may express a target sequence, preferably GMT using the regulatory sequences as disclosed herein. The present invention further encompasses not only such transgenic plants and portions thereof, but also transformed plant cells, including cells and seed of such plants, as well as progeny of such plants, for example produced from the seed.

In addition to their use in regulating nucleic acid expression, the regulatory sequences and fragments thereof of the present invention also have utility as probes in nucleic acid hybridization experiments to determine the presence of a sequence upstream of one of the GMT family members in a sample. Methods for preparing and using probes are described, for example, in Molecular Cloning: A Laboratory Manual, 2nd ed., vol. 1-3, ed. Sambrook et al., Cold Spring Harbor Laboratory Press, Cold Spring Harbor, N.Y., 1989 (“Sambrook et al., 1989”); Current Protocols in Molecular Biology, ed. Ausubel et al., Greene Publishing and Wiley-Interscience, New York, 1992 (with periodic updates) (“Ausubel et al., 1992); and Innis et al., PCR Protocols: A Guide to Methods and Applications, Academic Press: San Diego, 1990. Probes based on the regulatory sequences disclosed herein can be used to confirm and, if necessary, to modify the disclosed sequences by conventional methods, e.g., by re-cloning and re-sequencing.

The nucleic-acid probes of the present invention can hybridize under stringent conditions to a target DNA sequence. The term “stringent hybridization conditions” is defined as conditions under which a probe or primer hybridizes specifically with a target sequence(s) and not with non-target sequences, as can be determined empirically. The term “stringent conditions” is functionally defined with regard to the hybridization of a nucleic-acid probe to a target nucleic acid (i.e., to a particular nucleic-acid sequence of interest) by the specific hybridization procedure (see e.g., Sambrook et al., 1989, at 9.52-9.55 and 9.47-9.52, 9.56-9.58; Kanehisa, Nucl. Acids Res. 12:203-213, 1984; and Wetmur and Davidson, J. Mol. Biol. 31:349-370, 1968). As is well known in the art, stringency is related to the Tₘ of the hybrid formed. The Tₘ (melting temperature) of a nucleic acid hybrid is the temperature at which 50% of the bases are base-paired. For example, if one the partners in a hybrid is a short oligonucleotide of approximately 20 bases, 50% of the duplexes are typically strand separated at the Tₘ. In this case, the Tₘ reflects a time-independent equilibrium that depends on the concentration of oligonucleotide. In contrast, if both strands are longer, the Tm corresponds to a situation in which the strands are held together in structure possibly containing alternating duplex and denatured regions. In this case, the Tm reflects an intramolecular equilibrium that is independent of time and polynucleotide concentration.

As is also well known in the art, Tₘ is dependent on the composition of the polynucleotide (e.g. length, type of duplex, base composition, and extent of precise base pairing) and the composition of the solvent (e.g. salt concentration and the presence of denaturants such formamide). On equation for the calculation of Tₘ can be found in Sambrook et al. (Molecular Cloning, 2nd ed., Cold Spring Harbor Press, 1989) and is:

Tₘ=81.5° C.−16.6(log₁₀[Na^{+])=}0.41(% G+C)−0.63(% formamide)−600/L)

Where L is the length of the hybrid in base pairs, the concentration of Na⁺is in the range of 0.01M to 0.4M and the G+C content is in the range of 30% to 75%. Equations for hybrids involving RNA can be found in the same reference. Alternative equations can be found in Davis et al., Basic Methods in Molecular Biology, 2nd ed., Appleton and Lange, 1994, Sec 6-8.

Methods for hybridization and washing are well known in the art and can be found in standard references in molecular biology such as those cited herein. In general, hybridizations are usually carried out in solutions of high ionic strength (6×SSC or 6×SSPE) at a temperature 20-25° C. below the Tₘ. High stringency wash conditions are often determined empirically in preliminary experiments, but usually involve a combination of salt and temperature that is approximately 12-20° C. below the Tₘ. One example of high stringency was conditions is 1×SSC at 60° C. Another example of high stringency wash conditions is 0.5×SSPE, 0.1% SDS at 42° C. (Meinkoth and Wahl, Anal. Biochem., 138:267-284, 1984). An example of even higher stringency wash conditions is 0.1×SSPE, 0.1% SDS at 50-65° C. In one preferred embodiment, high stringency washing is carried out under conditions of 40% formamide, IM NaCl, 0.5% SDS, 5× Denhardts, 0.05 M NaPO4 buffer, pH 7.0, 0.08 mg/ml herring sperm DNA and 0.1 g/ml dextran sulphate at 42° C. overnight, followed by two washes of 0.5×sodium chloride/sodium citrate (SSC) at about 55° C. for 40 minutes. However, as is well recognized in the art, various combinations of factors can result in conditions of substantially equivalent stringency. Such equivalent conditions are within the scope of the present invention.

Accordingly, in one preferred embodiment, the nucleic acid sequences, SEQ ID NOS: 1-3, fragments, and complements thereof may be used as probes in assays of other plant tissues to identify closely related or homologous genes and associated regulatory sequences. These include southern hybridization assays on any substrate including but not limited to an appropriately prepared plant tissue, cellulose, nylon, or combination filter, chip, or glass slide. Such methodologies are well known in the art and are available in a kit or preparation that can be supplied by commercial vendors. Preferably, these assays will be used in methods to determine the presence of a sequence upstream of a sequence encoding GMT in a sample from Brassica napus. Such methods include the steps of contacting the sample with a nucleic acid probe which hybridizes to a nucleic acid molecule having the sequence of SEQ ID NO: 1; and determining whether the nucleic acid probe hybridizes to a nucleic acid molecule in said sample. In another preferred embodiment, the nucleic acid probe used hybridizes to a nucleic acid molecule having the sequence of SEQ ID NO: 2. In yet another preferred embodiment, the nucleic acid probe used hybridizes to a nucleic acid molecule having the sequence of SEQ ID NO: 3. Preferably, SEQ ID NO: 1-3 are located upstream of the sequence encoding for GMT.

The following examples are included to demonstrate preferred embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1

### Sequence Identification

The sequence of the Arabidopsis gamma-tocopherol methyl transferase gene (GenBank accession number AF104220) is used as a query sequence against a database of Expressed Sequence Tags (EST) sequences derived from the cDNA libraries prepared from various Brassica tissues using the BLASTN program. BLASTN parameters are set as follows: Number of alignments to show (B): 10; Number of one-line descriptions (V): 10; Expectation value (E): 10.0; Filter sequence query: Yes; Cost to open gap: 0; Cost to extend gap: 0; X dropoff value for gapped alignment: 0; Penalty for nucleotide mismatch: −3; Reward for a nucleotide match: 1; Threshold for extending hits: 0; Perform gapped alignment: Yes; Query Genetic code to use: Standard; DB Genetic code: Standard

A partial EST is identified from a 30 day after pollenation (DAP) Brassica napus silique library. This EST is LIB4153-002-Q1-K1-E3 which has an identity of 608/700(86%) and gaps equal to 9/700(1%).

### Example 2

### Genomic Library Construction. PCR Amplification and Sequence Isolation

To identify the sequences upstream of the Brassica napus GMT coding region, a genomic DNA library is prepared. A number of methods are known to those of skill in the art for genomic library preparation. For genomic libraries of the present invention, Brassica napus DNA (Quantum variety) leaves are isolated by commercially available Plant DNAzol® reagents according to kit instructions (Gibco BRL, Life Technologies, Gaithersburg, Md.). The libraries are prepared according to manufacturer instructions (GENOME WALKER™ (CLONTECH Laboratories, Inc, Palo Alto, Calif.) CLONTECH protocol number PT1116-1 version PR9Y596 published Nov. 10, 1999). In separate reactions, genomic DNA is subjected to restriction enzyme digestion overnight at 37° C. with the following blunt-end endodnucleases: EcoRV, ScaI, DraI, PvuII, or StuI (CLONTECH Laboratories, Inc. Palo Alto, Calif.). The reaction mixtures are extracted with phenol:chloroform, ethanol precipitated and resuspended in Tris-EDTA buffer. The purified blunt-ended genomic DNA fragments are then ligated to the GenomeWalker™ adaptors and ligation of the resulting DNA fragments to adaptors is performed according to manufacturer's protocol. The GenomeWalker™ sublibraries are aliquoted and stored at −20° C.

Genomic DNA ligated to the GenomeWalker™ adaptor as prepared above is subjected to a primary round of PCR amplification with gene-specific primer 1 (GSP1) and with a primer that anneals to the Adaptor sequence, adaptor primer 1 (AP1) which is provided with the kit. A diluted (1:50) aliquot of the primary PCR reaction is used as the input DNA for a nested round of PCR amplification with gene-specific primer 2 (GSP2) and with adaptor primer 2 (AP2) which is provided with the kit. Generally, gene specific primers are designed to have the following characteristics: 26-30 nucleotides in length, GC content of 40-60% with resulting temperatures for most of the gene specific primers in the high 60° C. range or about 70° C. Advantage® genomic polymerase mix (a mixture of Tth and Vent polymerase), available through Clontech, is the polymerase used. A number of temperature cycling instruments and reagent kits are commercially available for performing PCR experiments and include those available from PE Biosystems (Foster City, Calif.), Stratagene (La Jolla, Calif.), and MJ Research Inc. (Watertown, Mass.).

Primary PCR components and conditions generally used are as follows. For the primary PCR reactions, 1 μl of sub-library aliquot is combined with 1 μl (100 pmol) of Gene-specific primer 1, 1 μl of GenomeWalker™ Adaptor primer 1 (AP 1), 2.5 μl of dNTP mix (100×), 5 μl (final concentration of 1×) of 10× PCR buffer (containing MgCl₂), 0.5 μl of Advantage® genomic polymerase mix, and distilled water for a final reaction volume of 50 μl. Primary PCR reaction conditions are generally as follows: Step 1: 94° C. for 2 seconds, 72° C. for 3 minutes; repeat 94° C./72° C. cycling for total of 7 cycles; Step 2: 94° C. for 2 seconds, 67° C. for 3 minutes; repeat 94° C/67° C. cycling for total of 32 cycles; Step 3: 67° C. for 4 minutes as a final extension; and Step 4: 4° C. for an extended incubation.

Secondary PCR (nested PCR) components and conditions generally used are as follows. For the secondary PCR reactions, 1 μl of a 1:50 dilution of the primary PCR reaction is combined with 1 μl (100 pmol) of Gene-specific primer 2, 1 μl of GenomeWalker™ Adaptor primer 2 or 3 (AP2 or AP3), 2.5 μl of dNTP mix (100×), of 10× PCR buffer (containing MgCl₂) (to a final concentration of 1×), 0.5 μl of Advantage® genomic polymerase mix, and distilled water to a final reaction volume of 50 μl. Secondary (nested) PCR reaction conditions are generally as follows: Step 1: 94° C. for 2 seconds, 72° C. for 3 minutes; repeat 94° C/72° C. cycling for total of 5 cycles; Step 2: 94° C. for 2 seconds, 67° C. for 3 minutes; repeat 94° C/67° C. cycling for total of 20 cycles; Step 3: 67° C. for 4 minutes as a final extension; and Step 4: 4° C. for an extended incubation.

2a. Clone ID Analysis

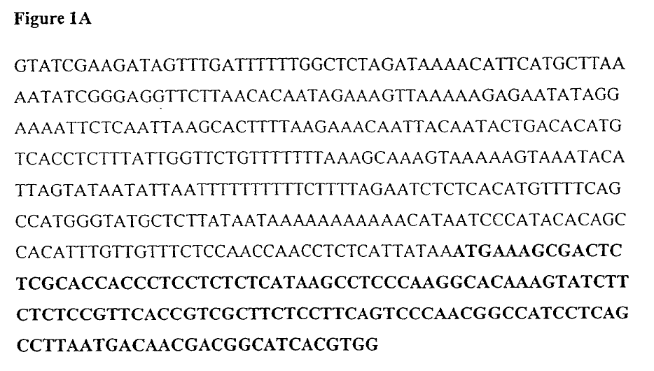

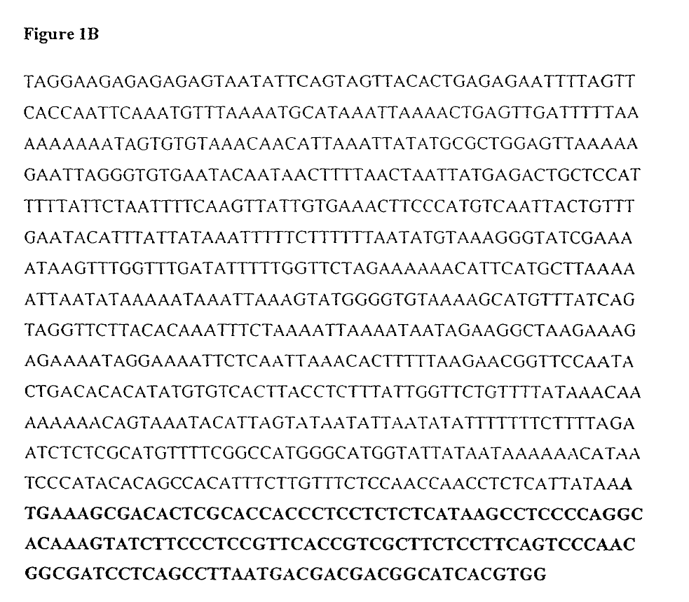

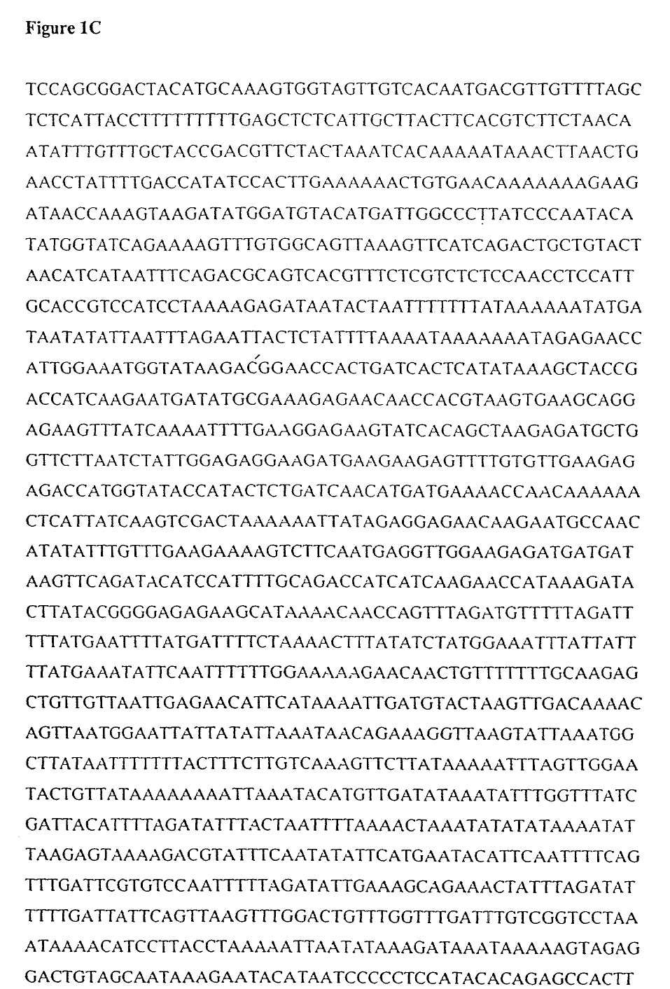

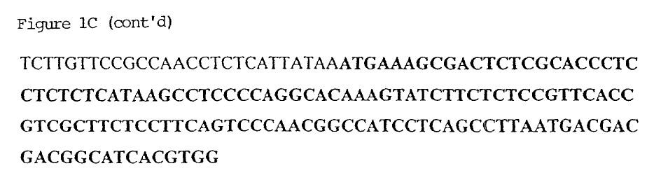

The following pair of gene specific primers for use with GenomeWalker™ were designed from the sequence of LIB4153-002-Q1-K1-E3. (E3-GSP1 5′GTGATGCATATGATCTCCCCAAATCTC3′ (SEQ ID NO: 10); E3-GSP2 5′CCACGTGATGCCGTCGTCGTCATTAAG3′ (SEQ ID NO: 11)) This set of primers is used with each of the libraries detailed above. Five μl of the PCR products from each these GenomeWalker™ PCR reactions is cloned into pCR2.1Topo (Invitrogen) as per the manufacturer's directions. A total of three clones are obtained which contain sequences upstream from one of the Brassica GMT coding regions. One clone is obtained from the EcoRV library (RV2.1 clone), one is from the PvuII digested library (pMON67501), and one is from the StuI library (pMON67502). Double stranded DNA sequence is obtained of the inserts in these three clones. The nucleic acid sequence of the three clones is as shown in FIGS. 1A, 1B and 1C. These three clones, encoding three distinct upstream sequences, support the fact that the GMT gene is represented at least three times in the Brassica napus genome.

### Example 3

### Southern Blot

DNA samples (10 μg) are digested to completion with restriction endonucleases according to instructions supplied by the vendor (Boehringer Mannheim Biochemicals, Indianapolis, Ind.). One sixth volume of loading buffer (0.25% bromophenol blue, 40% sucrose in H₂O) is added to each sample before loading onto 0.8% agarose gels. Gels are electrophoresed for approximately 16 hours at 45 V, photographed, and prepared for transfer to 0.45 nm nylon membranes (Nytran SuperCharge, Schleicher & Schuell, Keene, N.H.). Preparations for transfer consisted of gentle shaking for in 8 minutes in 10 ml HCl and 390 ml H₂O, a brief water rinse, shaking in a denaturing solution (Sambrook et al. 1989) for 45 minutes, shaking in a neutralizing solution (Sambrook et al. 1989) for 1-2 minutes, and a water rinse. DNA in the gels is then transferred to membranes overnight for 18 hours by capillary action using 10×SSC (Sambrook et al., 1989). Following transfer, the nylon membranes are crosslinked by UV using the autostratalink setting of a Stratalinker (Stratagene, Inc., La Jolla, Calif.) and then pre-hybridized for 2 hours at 42° C. in 25 ml of a solution containing 40% formamide, 1M NaCl, 0.5% SDS, 5× Denhardts, 0.05M NaPO4 buffer, pH 7.0, 0.08 mg/ml herring sperm DNA, and 0.1 g/ml dextran sulphate. The membranes are hybridized overnight in solutions identical to those described for pre-hybridizations, with the exception that the hybridization solutions also contain a denatured hybridization probe (the Sal/Not fragment of the EST LIB4153-002-Q1-K1-E3 which contains almost the entire coding region of the Brassica GMT) which has been radiolabeled with [symbol] P³²-dCTP by the random primer method (Oligolabeling Kit, Pharmacia, Peapack, N.J.). After hybridization the filter is rinsed several times at room temperature and then washed twice in a large volume of 0.5×SSC, 0.5% SDS at 55° C. for 40 minutes each. The membranes are then wrapped in plastic wrap and exposed to a phosphorimager screen for 2 hours. The screen is then scanned using the STORM 860 phosphorimager system (Molecular Dynamics, Inc., Sunnyvale, Calif.).

Southern blot, showing DNA digested individually with BamHI, EcoRI, or HindIII, evidences the existence of four GMT genes present in the Brassica napus genome by exhibiting at least 4 bands present in each lane.

### Example 4

### Transgenic Expression of Zinc Finger Protein Transcription Factors Designed to Bind to the Sequences Upstream of Gmt and Activate the Expression of the Gene

A zinc finger is one of the major structural motifs involved in eukaryotic protein-nucleic acid interaction. One extensively studied zinc finger protein is the transcription factor IIIA (TFIIIA) which contains a sequence motif of X₃-Cys-X₂₋₄-Cys-X₁₂-His-X₃₋₄-His-X₄ (where X is any amino acid). TFIIA-like zinc fingers contain an antiparallel β ribbon and an α helix. The two invariant cysteines, which are near the turn in the β ribbon region, and the two invariant histidines, which are in the COOH-terminal portion of the α helix coordinate a central zinc ion, and the finger forms a compact globular domain. Each Cys₂-His₂ zinc finger domain typically binds 3 base pairs of a double-stranded DNA sequence. Of the DNA binding motifs that have been manipulated by design or selection, the TFIIIA-related Cys₂-His₂ zinc finger proteins have demonstrated the greatest potential for manipulation into general and specific transcription factors.

Zinc finger proteins and in particular zinc finger transcription factors with novel DNA binding specificities can be obtained using phage display and affinity selection (Rebar and Pabo (1994) Science 263:671-673). Methods for the construction of phage display libraries are well known in the art and can be found, for example. In Smith and Petrenko (1997) Chem Rev. 97:391-410 and Lowman (1997) Ann. Rev. Biophys. Biomol. Struct. 26:401-424. In this procedure, zinc finger transcription factors are expressed on the surface of filamentous phage. In particular, polynucleotide sequences encoding transcription factors are introduced into the phage gene III and displayed as part of the gene III protein at one tip of the virion. In order to find transcription factors that bind specific DNA sequences, random mutations are introduced during synthesis of the DNA encoding the variable regions of the transcription factor. Methods for the synthesis of polynucleotides have been discussed previously. These sequences containing randomized mutations are then introduced into a filamentous phage and the phage library grown using well known procedures (Smith and Scott, (1993) Methods in Enzymology, 217:228). Phage displaying the novel zinc finger proteins are then affinity selected based on their ability to bind to the DNA sequence of interest. Commonly, selected phage are expanded and subjected to additional rounds of affinity selection. Phage which show the greatest binding specificity and affinity are then grown in culture, the DNA encoding the zinc finger protein isolated, and the DNA sequenced. If desired, additional specificity can be obtained by combining several zinc finger domains by the use of linker peptides. Methods for the production of such polydactyl zinc finger proteins are known in the art (Liu et al. (1997) Proc. Natl. Acad. Sci. USA 94:5525-5530 and Wang and Pabo (1999) Proc. Natl. Acad. Sci. USA 96:9568-9573).

It is to be understood that the present invention has been described in detail by way of illustration and example in order to acquaint others skilled in the art with the invention, its principles, and its practical application. Further, the specific embodiments of the present invention as set forth are not intended as being exhaustive or limiting of the invention, and that many alternatives, modifications, and variations will be apparent to those skilled in the art in light of the foregoing examples and detailed description. Accordingly, this invention is intended to embrace all such alternatives, modifications, and variations that fall within the spirit and scope of the following claims. While some of the examples and descriptions above include some conclusions about the way the invention may function, the inventors do not intend to be bound by those conclusions and functions, but puts them forth only as possible explanations.

We claim:

1. An isolated nucleic acid molecule comprising a nucleic acid sequence selected from the group consisting of SEQ ID NO: 1, SEQ ID NO: 2, SEQ ID NO: 3 and complements thereof.

2. The isolated nucleic acid molecule according to claim 1, wherein said nucleic acid sequence is SEQ ID NO: 1.

3. The isolated nucleic acid molecule according to claim 1, wherein said nucleic acid sequence is SEQ ID NO: 2.

4. The isolated nucleic acid molecule according to claim 1, wherein said nucleic acid sequence is SEQ ID NO: 3.

5. An isolated nucleic acid molecule comprising a nucleic acid sequence that is at least 30 consecutive nucleotides of a nucleic acid sequence selected from the group consisting of SEQ ID NO: 1, SEQ ID NO: 2, SEQ ID NO: 3 and complements thereof.

6. The isolated nucleic acid molecule according to claim 5, wherein said nucleic acid sequence is at least 50 consecutive nucleotides of a nucleic acid sequence selected from the group consisting of SEQ ID NO: 1, SEQ ID NO: 2, SEQ ID NO: 3 and complements thereof.

7. The isolated nucleic acid molecule according to claim 6, wherein said nucleic acid sequence is at least 75 consecutive nucleotides of a nucleic acid sequence selected from the group consisting of SEQ ID NO: 1, SEQ ID NO: 2, SEQ ID NO: 3 and complements thereof.

8. The isolated nucleic acid molecule according to claim 7, wherein said nucleic acid sequence is at least 100 consecutive nucleotides of a nucleic acid sequence selected from the group consisting of SEQ ID NO: 1, SEQ ID NO: 2, SEQ ID NO: 3 and complements thereof.

9. The isolated nucleic acid molecule according to claim 8, wherein said nucleic acid sequence is at least 150 consecutive nucleotides of a nucleic acid sequence selected from the group consisting of SEQ ID NO: 1, SEQ ID NO: 2, SEQ ID NO: 3 and complements thereof.

10. The isolated nucleic acid molecule according to claim 9, wherein said nucleic acid sequence is at least 200 consecutive nucleotides of a nucleic acid sequence selected from the group consisting of SEQ ID NO: 1, SEQ ID NO: 2, SEQ ID NO: 3 and complements thereof.

11. The isolated nucleic acid molecule according to claim 10, wherein said nucleic acid sequence is at least 250 consecutive nucleotides of a nucleic acid sequence selected from the group consisting of SEQ ID NO: 1, SEQ ID NO: 2, SEQ ID NO: 3 and complements thereof.

12. A vector comprising a nucleic acid molecule comprising a nucleic acid sequence selected from the group consisting of SEQ ID NO: 1, SEQ ID NO: 2, SEQ ID NO: 3 and complements thereof operably linked to polypeptide encoding nucleic acid sequence.

13. A vector comprising a nucleic acid molecule comprising a nucleic acid sequence selected from the group consisting of SEQ ID NO: 1, SEQ ID NO: 2, SEQ ID NO: 3 and complements thereof operably linked to a heterologous nucleic acid sequence in manner where the complement of said heterologous nucleic acid sequence is expressed.

14. A host cell having a heterologous nucleic acid molecule that comprises a nucleic acid sequence selected from the group consisting of SEQ ID NO: 1, SEQ ID NO: 2, SEQ ID NO: 3 and complements thereof.

15. A host cell having a heterologous nucleic acid molecule that comprises a nucleic acid sequence that is at least 30 consecutive nucleotides of a nucleic acid sequence selected from the group consisting of SEQ ID NO: 1, SEQ ID NO: 2, SEQ ID NO: 3 and complements thereof.

16. A plant having a heterologous nucleic acid molecule that comprises a nucleic acid sequence selected from the group consisting of SEQ ID NO: 1, SEQ ID NO: 2, SEQ ID NO: 3 and complements thereof.

17. A plant having a heterologous nucleic acid molecule that comprises a nucleic acid sequence that is at least 30 consecutive nucleotides of a nucleic acid sequence selected from the group consisting of SEQ ID NO: 1, SEQ ID NO: 2, SEQ ID NO: 3 and complements thereof.

18. A method of screening for compounds capable of effecting the level of gamma-tocopherol methyltransferase expression comprising:
(a) providing a cell with a nucleic acid sequence selected from the group consisting of SEQ ID NO: 1, SEQ ID NO: 2, SEQ ID NO: 3 and complements thereof operably linked to a heterologous nucleic acid sequence in manner where the complement of said heterologous nucleic acid sequence is expressed;
(b) providing a test compound to said cell; and
(c) determining the level of said complement of said heterologous nucleic acid sequence or a polypeptide encoded by said heterologous nucleic acid sequence.

19. A method according to claim 18, wherein said heterologous sequence encodes a marker polypeptide.

20. A method according to claim 19, where said marker polypeptide is selected from the group consisting of GFP, GUS, LUX, antibiotic markers, and herbicide tolerance markers.

21. A method of determining the presence of a nucleic acid sequence of at least 200 consecutive nucleotides in a sample comprising:
(a) contacting the sample with a nucleic acid probe that hybridizes to a nucleic acid sequence having the sequence of SEQ ID NO: 1; and
(b) determining whether the nucleic acid probe hybridizes to a nucleic acid molecule in said sample.
